(19) United States
(12) Patent Application Publication
(10) Pub. No.: US 2025/0152917 A1
(43) Pub. Date: May 15, 2025

(54) CATHETER WITH ENHANCED NAVIGABILITY TO AVOID OPHTHALMIC ARTERY

(71) Applicant: Neuravi Limited, Galway (IE)

(72) Inventors: AnnaLisa SMULLIN, Galway (IE); Clodagh DUNNE, Galway (IE); Meadhbh PARSONS, Galway (IE)

(73) Assignee: Neuravi Limited, Galway (IE)

(21) Appl. No.: 18/389,369

(22) Filed: Nov. 14, 2023

(51) Int. Cl. A61M 25/00; A61B 17/22; A61M 25/01

(52) U.S. Cl. CPC A61M 25/0068; A61M 25/0155; A61B 2017/22079; A61M 2025/0036; A61M 2025/004; A61M 2025/0177

(57) ABSTRACT

A catheter comprises an elongated body, a tip, a central lumen, and a bump. The elongated body comprises a proximal end, a distal end, a longitudinal axis, and an elongated body outer diameter. The tip is disposed at the distal end of the elongated body along the longitudinal axis. The tip comprises an opening comprising a tip outer diameter smaller than the elongated body outer diameter, a neck proximal of the opening comprising the tip outer diameter, and a shoulder disposed between the neck and the elongated body. The central lumen extends from the proximal end to the distal end along the longitudinal axis through the tip. The bump extends longitudinally from the elongated body to proximate the opening and is partially circumferentially attached to at least a portion of the neck and the elongated body, forming an asymmetric cross section of the distal end.

### FIELD

The present invention generally relates to medical instruments, and more particularly, to a catheter with enhanced navigability.

### BACKGROUND

Various types of catheters have been developed for use during neurovascular interventions. No matter which specific type of catheter is selected, a catheter needs to be able to track through a specific anatomy to reach a treatment site. Then depending on the functionality of the selected catheter, an implant, another catheter, or other device (e.g., delivery aid) may go through inside its lumen to implement the designated task. For example, an aspiration catheter is known as an intermediate or distal access catheter. Such aspiration catheter can be used in mechanical thrombectomy procedures to perform, for example, a direct contact aspiration. In this case, an aspiration catheter navigates through the vascular anatomy to reach the occlusion site, with its proximal end connected to a syringe or aspiration pump which generates negative pressure to engage or ingest the thrombus.

During the process of navigating through the vasculature of a patient, factors such as efficiency and navigability are main factors to evaluate a performance of a catheter. Navigability is a desired character especially for distal access catheters (e.g., to ease navigation and effective aspiration). However, this can be challenging due to the nature of the vasculature in which the catheter is navigating. Therefore, there is always a need for improved or enhanced navigability for various catheters.

### SUMMARY

Disclosed herein are various exemplary catheters with enhanced navigability, which may at least alleviate above needs. Also, a corresponding method for steering a catheter is provided in this disclosure.

According to an aspect of this disclosure, a catheter comprises an elongated body, a tip, a central lumen, and a bump. The elongated body comprises a proximal end, a distal end, a longitudinal axis, and an elongated body outer diameter. The tip disposed at the distal end of the elongated body along the longitudinal axis. The tip comprises an opening comprising a tip outer diameter smaller than the elongated body outer diameter, a neck proximal of the opening comprising the tip outer diameter, and a shoulder disposed between the neck and the elongated body. The central lumen extends from the proximal end to the distal end along the longitudinal axis through the tip. The bump extends longitudinally from the elongated body to proximate the opening and is partially circumferentially attached to at least a portion of the neck and the elongated body, forming an asymmetric cross section of the distal end.

In some examples, the asymmetric cross section comprises an outer diameter greater than an average inner diameter of an ophthalmic artery to prevent the catheter from entering the ophthalmic artery.

In some examples, the outer diameter of the asymmetric cross section is smaller than an average inner diameter of a cerebral vessel to allow the catheter to go through the cerebral vessel.

In some examples, the catheter further comprises a side lumen disposed within a sidewall of the catheter extending from the proximal end to the distal end along the longitudinal axis and exiting through the tip, configured to receive a guidewire.

In some examples, the central lumen comprises a uniform inner diameter.

According to another aspect of this disclosure, a catheter comprises an elongated body, a tip, a central lumen, and at least one steering lumen. The elongated body comprises a proximal end, a distal end, a longitudinal axis, and an elongated body outer diameter. The tip is disposed at the distal end of the elongated body along the longitudinal axis. The tip comprises an opening comprising a tip outer diameter smaller than the elongated body outer diameter, a neck proximal of the opening comprising the tip outer diameter, and a shoulder disposed between the neck and the elongated body. The central lumen extends from the proximal end to the distal end along the longitudinal axis through the tip. The at least one steering lumen extends within a sidewall of the catheter from the proximal end to the distal end along the longitudinal axis through the tip, the at least one steering lumen being closed proximate the opening.

In some examples, the at least one steering lumen comprises a steering lumen opening on a circumferential tip outer surface proximate the opening.

In some examples, the steering lumen opening extends substantially radially through the sidewall.

In some examples, the at least one steering lumen comprises a sealed distal end proximate the opening.

In some examples, the at least one steering lumen comprises at least one branch extending substantially radially inwardly with an inflated internal volume when its inside injected liquid is pressurized.

In some examples, the catheter comprises at least two steering lumens symmetrically disposed about a full circumference of the catheter.

In some examples, the catheter comprises at least four steering lumens.

In some examples, the catheter further comprises a side lumen disposed within a sidewall of the catheter extending from the proximal end to the distal end along the longitudinal axis and exiting through the tip, configured to receive a guidewire.

In some examples, the catheter further comprises a side lumen disposed within a sidewall of the catheter extending from the proximal end to the distal end along the longitudinal axis and exiting through the shoulder, configured to receive a guidewire.

In some examples, the catheter further comprises an inflatable annular balloon being full circumferentially attached to at least a portion of the elongated body, or a portion of the tip.

In some examples, the inflatable annular balloon when inflated comprises an outer diameter greater than an average inner diameter of an ophthalmic artery to prevent the catheter from entering the ophthalmic artery.

In some examples, the outer diameter of the inflatable annular balloon when inflated is smaller than an average inner diameter of a cerebral vessel, to allow the catheter to go through the cerebral vessel.

According to a further aspect of this disclosure, a method for steering a catheter. The catheter comprises an elongated body, a tip, a central lumen, and at least one steering lumen. The elongated body comprises a proximal end, a distal end, a longitudinal axis, and an elongated body outer diameter. The tip is disposed at the distal end of the elongated body along the longitudinal axis. The tip comprises an opening comprising a tip outer diameter smaller than the elongated body outer diameter, a neck proximal of the opening comprising the tip outer diameter, and a shoulder disposed between the neck and the elongated body. The central lumen extends from the proximal end to the distal end along the longitudinal axis through the tip. The at least one steering lumen extends within a sidewall of the catheter from the proximal end to the distal end along the longitudinal axis through the tip, the at least one steering lumen being closed proximate the opening. The method comprising selecting a steering lumen from the at least one steering lumen, injecting a liquid into the selected steering lumen, pressurizing the selected steering lumen, and steering the tip of the catheter.

In some examples, the method further comprises injecting saline into the selected steering lumen.

Other aspects and features of the present disclosure will become apparent to those skilled in the pertinent art, upon reviewing the following detailed description in conjunction with the accompanying figures.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is an illustration of an exemplary distal portion of a catheter according to aspects of the present invention;

FIG. 2A is an illustration of an exemplary distal portion of the catheter as illustrated in FIG. 1 having a bump attached thereto according to aspects of the present invention;

FIG. 2B is an illustration of a cross-sectional view of the exemplary distal portion of the catheter taken along line 2-2 of FIG. 2A and looking in the direction of the arrows according to aspects of the present invention;

FIG. 3 is an illustration of a cross-sectional view of the catheter taken along line 3-3 of FIG. 2A and looking in the direction of the arrows according to aspects of the present invention;

FIGS. 4A to 4B are illustrations of the catheter as illustrated in FIG. 2A navigating through a vascular system according to aspects of the present invention;

FIGS. 5A to 5C are illustrations of exemplary scenarios of attaching a bump to the catheter as illustrated in FIG. 1 according to aspects of the present invention;

FIG. 6A is an illustration of an exemplary distal portion of the catheter as illustrated in FIG. 1 having a side lumen according to aspects of the present invention;

FIG. 6B is an illustration of a cross-sectional view of the exemplary distal portion of the catheter taken along line 6B-6B of FIG. 6A and looking in the direction of the arrows according to aspects of the present invention;

FIG. 6C is an illustration of a perspective view of the exemplary distal portion of the catheter as illustrated in FIG. 6A according to aspects of the present invention;

FIG. 6D is cross-sectional view of another exemplary distal portion of the catheter taken along line 6D-6D of FIG. 1 and looking in the direction of the arrows, which has a side lumen according to aspects of the present invention;

FIG. 7A is an illustration of a perspective view of an exemplary tip of a catheter having a steering lumen according to aspects of the present invention;

FIG. 7B is an illustration of a cross-sectional view of the exemplary tip of the catheter taken along line 7B-7B of FIG. 7A and looking in the direction of the arrows according to aspects of the present invention;

FIG. 7C is an illustration of a cross-sectional view of the exemplary tip of the catheter taken along line 7C-7C of FIG. 7A and looking in the direction of the arrows according to aspects of the present invention.

FIG. 7D is an illustration of cross-sectional views of the exemplary tip of the catheter taken along line 7D-7D of FIG. 7A and looking in the direction of the arrows according to aspects of the present invention;

FIG. 8 is an illustration of an implemental environment of the exemplary tip of the catheter as illustrated in FIG. 7A according to aspects of the present invention;

FIG. 9A is an illustration of a perspective view of another exemplary tip of a catheter having another type of steering lumen according to aspects of the present invention;

FIG. 9B is an illustration of a cross-sectional view of the exemplary tip of the catheter taken along line 9B-9B of FIG. 9A and looking in the direction of the arrows according to aspects of the present invention;

FIG. 9C is an illustration of a cross-sectional view of the exemplary tip of the catheter taken along line 9C-9C of FIG. 9A and looking in the direction of the arrows according to aspects of the present invention;

FIG. 10 is an illustration of an implemental scenario of the exemplary tip of the catheter as illustrated in FIG. 9A according to aspects of the present invention;

FIGS. 11A to 11D are illustrations of a plurality of exemplary scenarios of the exemplary catheter as illustrated in FIG. 1 having an inflatable annular balloon attached thereto according to aspects of the present invention;

FIG. 12 is an illustration of a rest status and inflated status of the exemplary inflatable annular balloon of the catheter as illustrated in FIG. 11A according to aspects of the present invention;

FIG. 13 is an illustration of an implemental scenario of the exemplary catheter having an inflatable annular balloon as illustrated in FIG. 11A according to aspects of the present invention;

FIG. 14 is a flow diagram illustrating exemplary method for steering a catheter according to aspects of the present invention.

### DETAILED DESCRIPTION

In general, various exemplary catheters described herein have enhanced navigability. With an improved navigating performance, the catheters (e.g., an aspiration catheter) can be more suitable for navigating through challenging anatomies. For example, the catheters may be easier to navigate through a cerebral vessel. Meanwhile, the catheters described in this disclosure may also be prevented from entering the ophthalmic artery. In some examples, the catheters with enhanced navigability may become more steerable with a steering lumen being designed inside its tip. In some other examples, the catheters with enhanced navigability may have a built-in guidewire with a side lumen being designed inside its tip. In this case, the catheters may reach a clot in a shorter period of time. More advantages of those catheters will be understood through detailed explanations below in conjunction with the drawings.

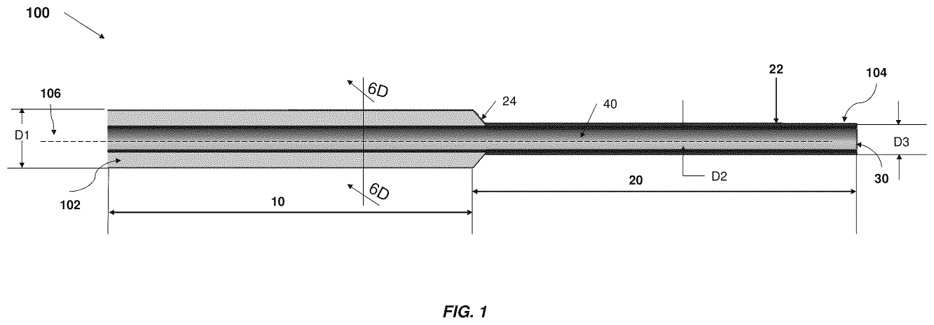

FIG. 1 illustrates an exemplary distal portion of a catheter. In FIG. 1, a catheter 100 may comprise an elongated body 10 whose outer diameter is D1. Herein, the elongated body 10 is shown as having a uniform outer diameter. However, it's to be understood that the catheter 100 is just an exemplary catheter among various catheters. Therefore, some catheters which may also be appropriate to implement examples described herein may have different shape and configurations (e.g., non-uniform outer diameter). The elongated body 10 comprises a proximal end 102, a distal end 104, and a longitudinal axis 106. At the distal end 104, outer shape of the catheter 100 may taper down, either gradually or relative sharply, to a smaller outer diameter. At such distal end 104, catheter 100 comprises a tip 20 which is disposed along the longitudinal axis 106. Tip 20 comprises an opening 30 whose outer diameter is D2. Herein, the outer diameter D2 of tip 20 is smaller than the elongated body's outer diameter D1. As an example, the smaller outer diameter D2 may be as thin as 15 mm. The tip 20 also comprises a neck 22 proximal of opening 30. Tip 20 also comprises a shoulder 24, which is disposed between neck 22 and the elongated body 10. Tip 20 tapers down in diameter from its proximal end adjacent to elongated body 10 to its distal end adjacent to neck 22. Catheter 100 further comprises a central lumen 40 which extends from the proximal end 102 to the distal end 104 along the longitudinal axis 106 through the tip 20. Herein, the central lumen 40 is illustrated as having a uniform inner diameter D3. However, it's to be understood that depending on the specific type of a catheter and its functionalities, a uniform inner diameter of a central lumen is not a necessity for applying various examples described in this disclosure to the catheter.

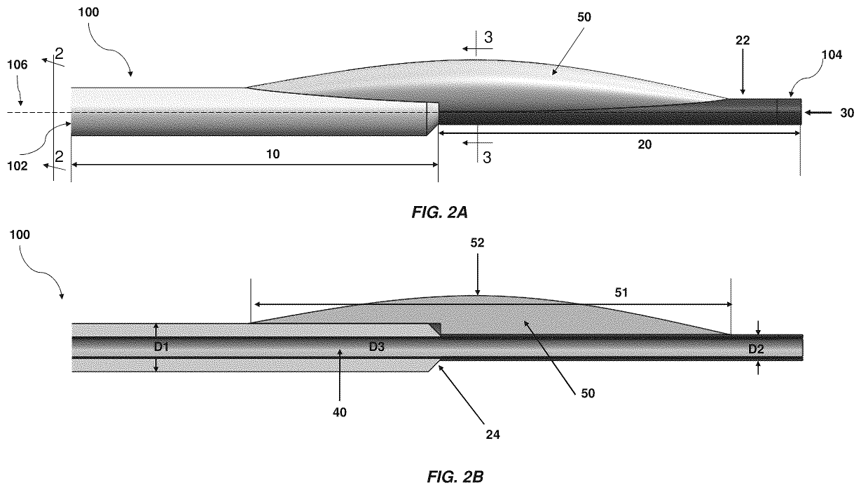

FIG. 2A illustrates attaching a bump 50 to the catheter in FIG. 1. FIG. 2B is a cross-sectional view of the catheter taken along line 2-2 of FIG. 2A and looking in the direction of the arrows. FIG. 3 is a cross-sectional view of the catheter taken along line 3-3 of FIG. 2A and looking in the direction of the arrows. In FIGS. 2A and 2B, a bump 50 is attached to one side of catheter 100. In other words, bump 50 is partially circumferentially attached to the catheter. Therefore, referring to FIG. 3, a cross-sectional view of the distal end 104 has an asymmetric cross section 60. In FIG. 3, the asymmetric cross section 60 has an outer diameter D5. Referring back to FIGS. 2A and 2B, bump 50 has a length 51, and extends longitudinally from the elongated body 10 to proximate the opening 30. In FIGS. 2A and 2B, bump 50 covers a portion of the elongated body 10, the whole shoulder 24, and a portion of neck 22. In this way, bump 50 covers a portion of catheter 100 which transitions from a larger outer diameter D1 to a smaller outer diameter D2. Bump 50 allows the tip 20 to steer around bends in the vasculature. Therefore, the bump 50 may cause the tip 20 and therefore the catheter 100 to navigate easier around bends, avoiding entering a wrong vasculature path where it's not desired to enter.

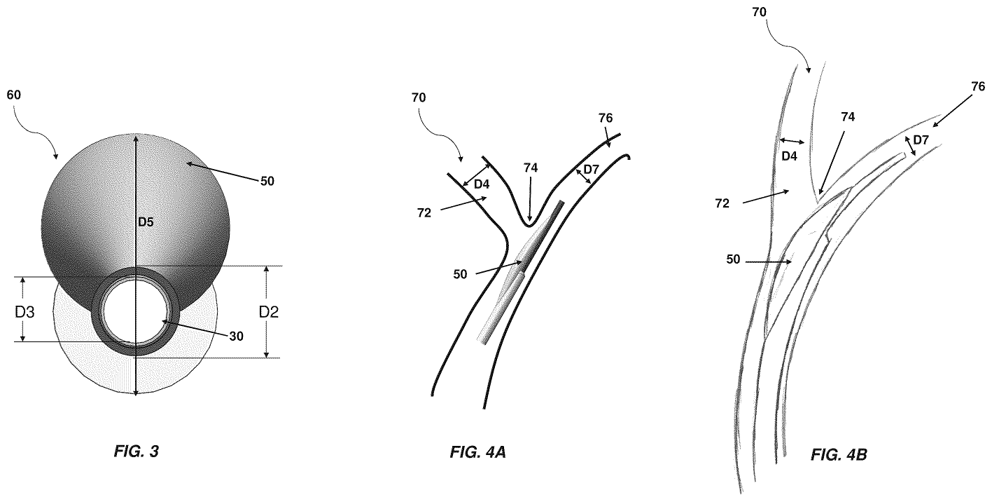

FIGS. 4A and 4B illustrate scenarios where the catheter 100 navigates through a vascular system 70. As illustrated in FIGS. 4A and 4B, the vascular system 70 comprises a cerebral vessel 76, wherein an ophthalmic artery 72 branches from the cerebral vessel 76 at a split point 74. Because bump 50 is attached to the distal end 104 of catheter 100, it is easier for catheter 100 to steer around the bifurcation 74 and bypass by the ophthalmic artery 72. Moreover, bump 50 may also cause the tip 20 to deform and therefore obtain a bended shape, as illustrated in FIG. 4B. Such bended shape may further assist tip 20 when being navigated around bends, branches, or the like (e.g., the branch formed around the bifurcation 74). Herein, to navigate through the cerebral vessel 76, the outer diameter D5 of the asymmetric cross section 60 is smaller than the inner diameter D7 of a cerebral vessel 76. In this case, outer diameter D5 is preferably designed to be smaller than an average inner diameter D7 of a human cerebral vessel 76.

In some examples, to keep the tip 20 from entering the ophthalmic artery 72, the outer diameter D5 of the asymmetric cross section 60 may be designed as greater than an inner diameter D4 of an ophthalmic artery 72. For example, the outer diameter D5 may be designed as smaller than an average inner diameter D4 of a human ophthalmic artery 72. In this way, the tip 20, and accordingly the catheter 100, may be prevented from entering the ophthalmic artery 72. For example, the mean inner diameter of the ophthalmic artery for men is 1.43±0.24 mm, and 1.34±0.20 mm for women. Considering there is no statistically significant difference in diameter for gender or age, a same outer diameter D5 may be used for various patients. But it's to be understood that such parameter may be designed differently as needed, to accommodate for different medical needs of different ages and genders if there are variations in the size of the ophthalmic artery so long as D5 is larger than the diameter of the ophthalmic artery. As an example, the mean average inner diameter D4 can be 1.38±0.23 mm at an entrance to an optical vessel, which is measured at about 5 mm from origin.

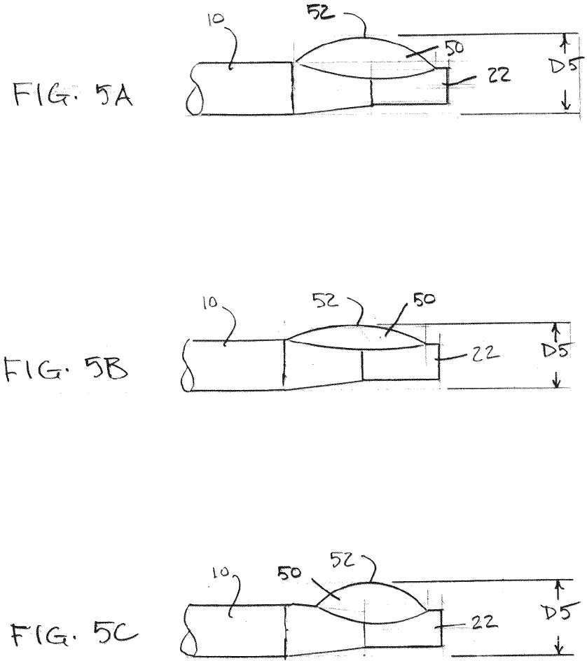

FIGS. 5A to 5C illustrates three exemplary scenarios of attaching a bump 50 to the catheter 100. As illustrated in FIGS. 5A and 5B, a bump 50 may be designed as having different outer diameters. Therefore, the outer diameter D5 of the asymmetric cross section 60 may vary within a certain range (e.g., greater than D4 but smaller than D7). Herein, the outer diameter D5 is measured from a top 52 of the bump 50 to the bottom, or opposite diametric side, of the elongated body 10, as illustrated in FIGS. 3 and 5A-5C. In some scenarios, instead of covering a portion of the elongated body 10, the whole shoulder 24 and a portion of the neck 22, bump 50 may instead cover a portion of the shoulder 24 and a portion of the neck 22, as illustrated in FIG. 5C.

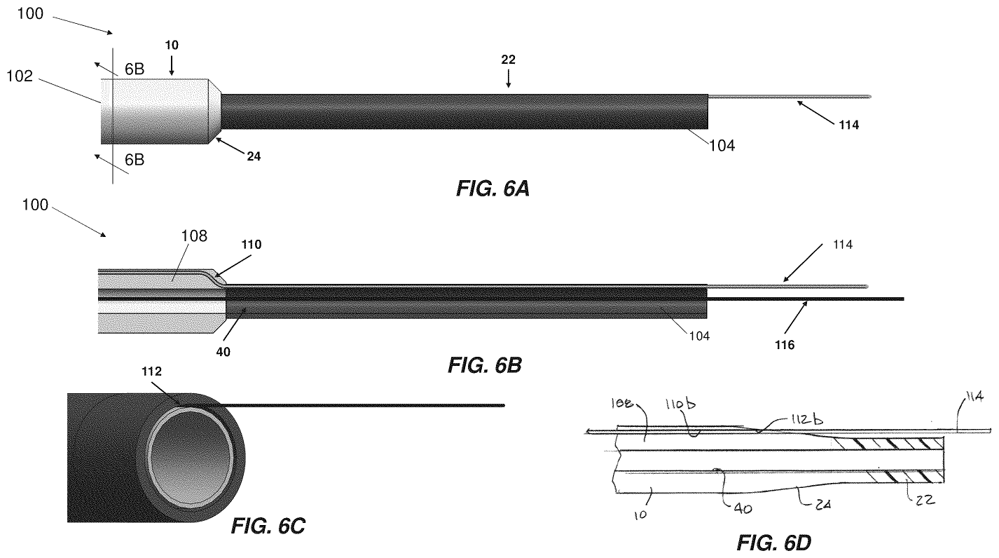

FIG. 6A illustrates another exemplary embodiment of the distal portion of catheter 100 in FIG. 1, FIG. 6B illustrates a cross-sectional view of the distal portion of the catheter taken along line 6B-6B of FIG. 6A and looking in the direction of the arrows, FIG. 6C is a perspective view of the distal portion of the distal portion of the catheter in FIG. 6A. As shown in FIGS. 6A-6C, a side lumen 110 is disposed within sidewall 108 of catheter 100, extending from the proximal end 102 to the distal end 104 along the longitudinal axis 106 (which is not shown in FIGS. 6A-6C). The side lumen 110 exits through the tip 20 with an opening 112 at the distal end surface of tip 20. The side lumen 110 is configured to receive a guidewire 114. In this case, catheter 100 has dual lumens. Guidewire 114 can act as a spine to the catheter 100 and thereby the whole catheter is able to be steered and torqued. Also, with guidewire 114 incorporated into the catheter 100, it will save time for a physician to install a guidewire into the catheter 100 on site. In this way, the time it takes to reach a clot may be reduced. Furthermore, the central lumen 40 is still available for a physician or an operator to insert a guidewire that he or she wants to use. Referring to FIG. 6B, as an example, a guidewire 116 may be inserted into the central lumen 40, which is a guidewire chose by the physician or operator. In FIGS. 6A-6C, the side lumen 110 tapers down along with the catheter 100. However, it's to be understood that the side lumen may also be all the way straight without a tapering down feature where the relevant outline of the sidewall of the catheter allows.

FIG. 6D illustrates a cross-sectional view of another exemplary embodiment of a distal portion of catheter 100 taken along line 6D-6D of FIG. 1 and looking in the direction of the arrows. In FIG. 6D, the side lumen 110b is disposed within the sidewall 108 of the catheter 100, extending from the proximal end 102 to the distal end 104 along the longitudinal axis 106. However, different from the embodiment illustrated in FIGS. 6A-6C, in FIG. 6D, the side lumen 110b exits through the shoulder 24 with an opening 112b. Similarly, the side lumen 110 is configured to receive the guidewire 114.

In order to not obscure the relevant features to be described, bump 50 is not shown in FIGS. 6A-6D. However, this does not mean that those features mutually exclude each other. To the contrary, a catheter having a bump attached to its distal portion may also have a side lumen. For example, the bump and the side lumen may be designed on the same side, or different sides of a catheter (e.g., catheter 100 described herein).

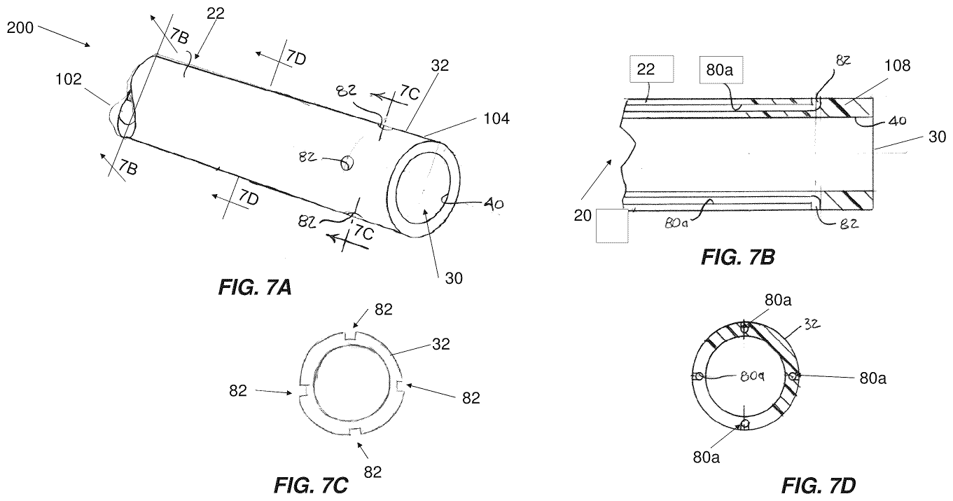

FIG. 7A is a perspective view of an exemplary tip 20 of a catheter 200. Herein, catheter 200 has the same components and therefore share the same reference numbers as catheter 100 from FIG. 1. Catheter 200 differs from catheter 100 in that, catheter 200 has a steering lumen. FIG. 7B is a cross-sectional view of the exemplary tip 20 of the catheter 200 taken along line 7B-7B of FIG. 7A and looking in the direction of the arrows. As illustrated in FIGS. 7A and 7B, steering lumens 80a extend within the sidewall 108 of the tip 22 of catheter 200, from the proximal end 102 to the distal end 104 along the longitudinal axis 106 through the tip 20. The steering lumens 80a are closed proximate the opening 30. Instead, referring to FIG. 7A, each steering lumen 80a has a steering lumen opening 82 on a circumferential tip outer surface 32 proximate the opening 30. Referring to FIG. 7B, the steering lumen openings 82 extend substantially radially through the sidewall 108. FIGS. 7C and 7D are cross-sectional views of the exemplary tip 20 of catheter 200 along line 7C-7C and line 7D-7D and looking in the direction of the arrows respectively. In the examples illustrated in FIGS. 7C and 7D, four steering lumens 80a are configured thereof, which are symmetrically disposed about a full circumference of the catheter 200.

It's to be understood that scenarios illustrated in FIGS. 7A-7D are merely exemplary examples, rather than any limitations to any examples described herein. Although four steering lumens are shown above, a catheter implementing an example of this disclosure may have only one steering lumen, or at least two steering lumens, or any appropriate number that meets specific needs. The steering lumens may be disposed symmetrically, or alternatively asymmetrically, or a combination thereof.

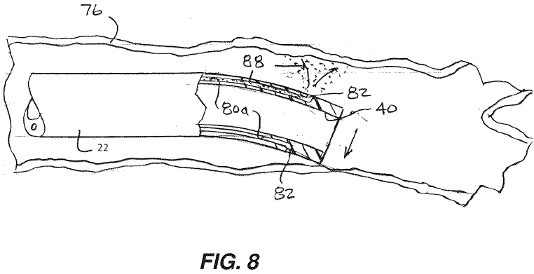

FIG. 8 illustrates an implemental environment of the exemplary tip 20 of catheter 200 as illustrated in FIG. 7A. With the additional steering lumens 80a configured inside the sidewall 108 of the catheter 200, liquid 88, such as, for example, saline may be injected into the internal steering lumens 80a. When liquid 88 is injected through a steering lumen 80a, it passes through the internal steering lumen 80a and exits the steering lumen 80a from the opening 82 in the sidewall of tip 20 of catheter 200. This process may cause the tip 20 to move away from the wall of cerebral vessel 76. For example, injecting liquid 88 into the upper steering lumen 80a as shown in FIG. 8 may cause the tip 20 of catheter 200 to move downward. Similarly, if liquid 88 is injected into the lower steering lumen 80a as shown in FIG. 8, the tip 20 of catheter 200 may be caused to move upward. Therefore, the steering lumen(s) 80a may allow a physician or an operator to steer the catheter 200 around tortuous bends in the vessels (e.g., the branch as shown in FIGS. 4A-4B).

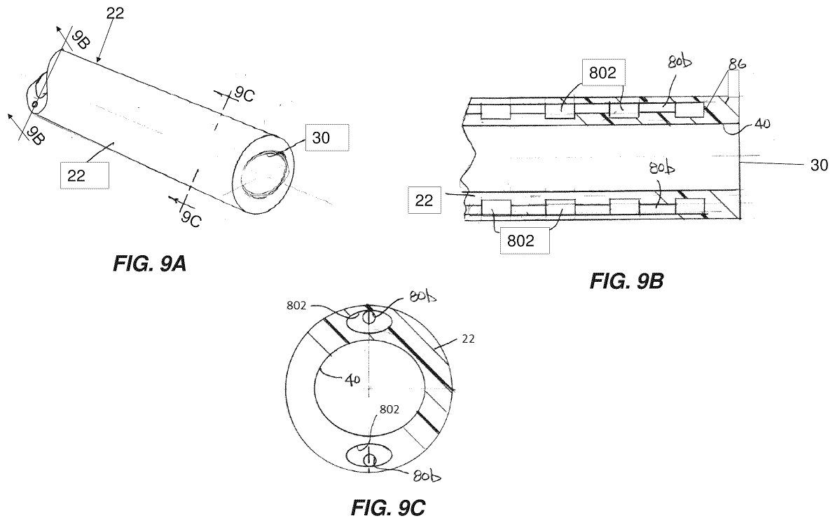

FIG. 9A is a perspective view of another exemplary tip 20 of a catheter 200 and FIG. 9B is a cross-sectional view taken along line 9B-9B of FIG. 9A and looking in the direction of the arrows. Similar to FIG. 7A, catheter 200 in FIG. 9A also has steering lumens 80b. However, each steering lumen 80b has a sealed distal end 86 proximate the opening 30. Therefore, referring to FIG. 9A, there are no openings on the outer surface of the neck 22. Referring back to FIG. 9B, the steering lumens 80b comprise branches 802 extending inwardly. In some examples, the branches 802 can extend substantially radially inwardly. Such steering lumen 80b forms a microchannel inside or internal to the catheter 200. Referring to FIG. 9C, which is a cross-sectional view along the 9C-9C line in FIG. 9A and looking in the direction of the arrows, two steering lumens 80b are configured inside the sidewall 108 of catheter 200. However, it's to be understood that any appropriate number of steering lumens 80b may be designed to meet the needs, such as one, two, three, four, or even more. Also, when more than one steering lumens 80b are deployed, they can be disposed symmetrically, asymmetrically, or a combination about a full circumference of the catheter 200. In some examples, the steering lumens 80a and 80b may be used together, as long as such design can meet the needs with at least the same or better performance than using either 80a or 80b.

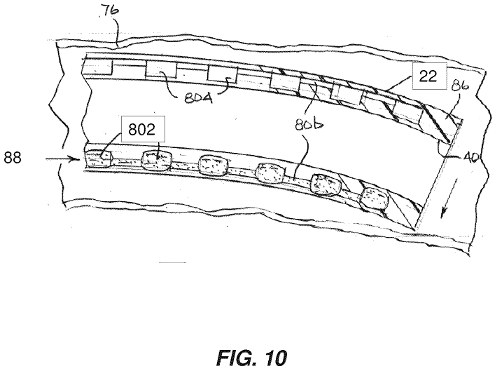

FIG. 10 illustrates an implemental scenario of the exemplary tip 20 of the catheter 200 as illustrated in FIG. 9A. Similar to the implemental scenario illustrated in FIG. 8, fluid 88 (e.g., saline) can be injected into a steering lumen 80b. When the liquid 88 is injected into steering lumen 80b, the side branches 802 disposed along the steering lumen 80b can expand. In other words, the branches can have an inflated internal volume when its inside injected liquid 88 is pressurized. In FIG. 10, branches 802 of the upper steering lumen 80b have a smaller internal volume than the branches 802 of the lower steering lumen 80b, because liquid 88 is injected into the lower steering lumen 80b and pressurized. The expansion of small branches 802 may cause the tip 20 of the catheter 200 to bend towards the side of the catheter or steering lumen 80b being injected, as shown in FIG. 10. For example, injecting liquid 88 into the lower steering lumen 80b may result in the tip 20 moving downward. Similarly, injecting liquid 88 into the upper steering lumen 80b may result in the tip 20 moving upward. To increase the degrees of freedom of the tip 20, more steering lumens 80b may be designed inside the catheter 200.

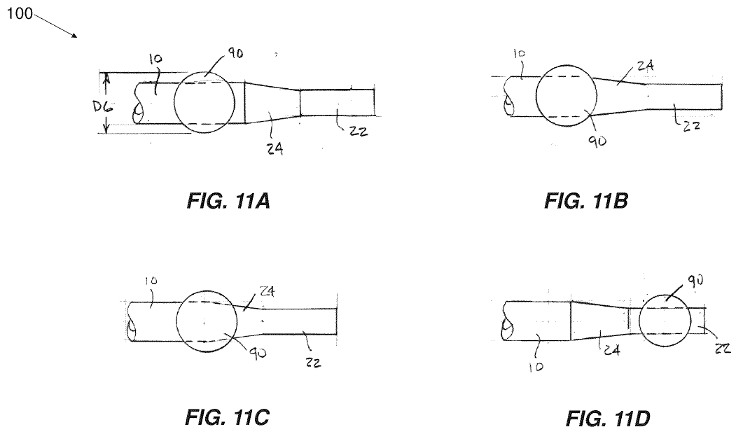

FIGS. 11A to 11D illustrate a plurality of exemplary scenarios of the exemplary catheter 100 as illustrated in FIG. 1, wherein the catheters illustrated herein have an inflatable annular balloon 90. Herein, the inflatable annular balloon 90 is attached around the full circumference of the catheter 100, to act as a guard for not entering the ophthalmic vessel 72. As illustrated in FIGS. 11A-11D, the inflatable annular balloon 90 may be attached to the catheter 100 and thereby cover different parts of its distal portion. As an example, referring to FIG. 11A, the inflatable annular balloon 90 may be attached to and covers only the elongated body 10 of catheter 100 with a certain predetermined distance from the shoulder 24. Alternatively, the inflatable annular balloon 90 may be located close to or covers a portion of the shoulder 24, as shown in FIGS. 11B and 11C, respectively. As another example, the inflatable annular balloon 90 may be attached to the neck 22 of the tip 20, as shown in FIG. 11D. In this regard, it's to be understood that such flexibility of a location of the inflatable annular balloon 90 may be desired, considering various types of catheters and therefore various configurations of the catheters.

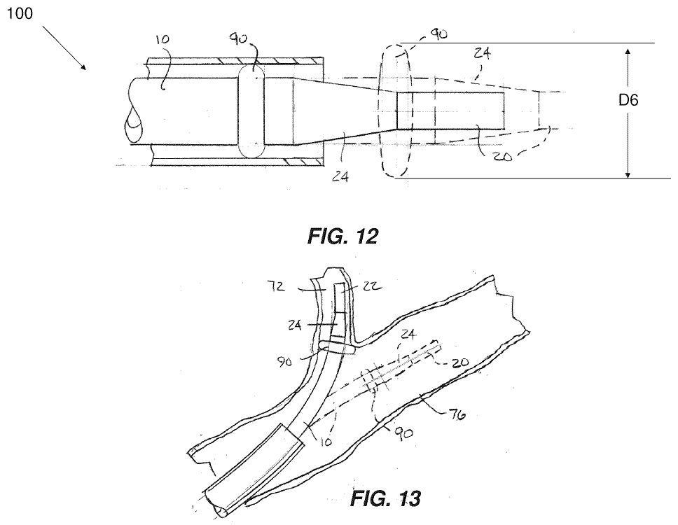

FIG. 12 is an illustration of a rest or uninflated state and inflated state of the exemplary inflatable annular balloon of the catheter as illustrated in FIG. 11A. Herein, the rest state of the inflatable annular balloon 90 may facilitate the catheter 100 to navigate through the vasculature of a patient. The inflated state of the inflatable annular balloon 90 may prevent the catheter from entering certain vessels (e.g., cerebral vessel).

FIG. 13 illustrates an implemental scenario of the exemplary catheter having an inflatable annular balloon 90. Herein, the inflatable annular balloon 90 has an outer diameter D6 when inflated. To allow the catheter 100 to go through the cerebral vessel 76, D6 is designed to be smaller than an inner diameter D7 of cerebral vessel 76. For example, D6 is designed as smaller than an average inner diameter D7 of a human cerebral vessel 76. Similarly, inflated balloon 90 will prevent catheter 100 from entering the ophthalmic artery 72. D6 is designed as greater than inner diameter D4, which may be an average inner diameter D4 of a human ophthalmic artery. In other words, the diameter of the inflatable annular balloon 90 may be based on typical dimensions of the ophthalmic artery so that the inflatable annular balloon 90 is larger than the ophthalmic artery and, therefore, won't enter the ophthalmic artery 72. As an example, the outer diameter D6 of the inflatable annular balloon can be about 1.65 mm±0.5 mm, which is larger than the mean ophthalmic diameter but still smaller than the mean cerebral vessel to allow a delivery aid to go through the catheter 100.

It's to be understood that although FIGS. 7A-13 merely shows a steering lumen or the inflatable annular balloon, various examples illustrated throughout all the drawings may be combined in any appropriate way to enhance a navigability of a tip of a catheter. It's also to be understood that the materials utilized and the way to attach a bump or an inflatable annular balloon to a catheter may depend on the characters or features of the catheter to implement various examples described herein. Any suitable manner could be chosen to fulfill the implementations in this disclosure.

Moreover, it to be understood that in this disclosure, vascular system 70, ophthalmic artery 72, and cerebral vessel 76 are described as exemplary implementations. Applications or implementations of various examples disclosed herein are not limited to those specific scenarios. For example, other vascular environment may be involved with different inner diameters. In this case, all the dimensions discussed herein (e.g., the outer diameter D5 of the bump 50, the outer diameter D6 of the inflatable annular balloon 90) may be designed differently to accommodate to the specific environment and meet implementation needs.

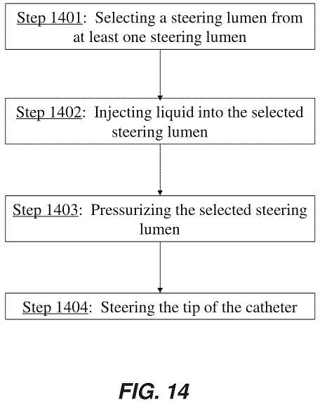

FIG. 14 is a flow diagram illustrating exemplary method for steering a catheter 200. The catheter may be any specific configurations or any combination thereof with at least one steering lumen 80a, 80b. At step 1401, select a steering lumen from the at least one steering lumens 80a, 80b. Then at step 1402, inject a liquid into the selected steering lumen 80a, 80b. At step 1403, pressurize the selected steering lumen 80a, 80b, and steer the tip 20 of the catheter 200 at step 1404. In some examples, the method may further comprise a step of injecting saline into the selected steering lumen 80a, 80b.

Aspects of the invention are also provided by the following numbered clauses:
Clause 1. A catheter comprising: an elongated body comprising a proximal end, a distal end, a longitudinal axis, and an elongated body outer diameter; a tip disposed at the distal end of the elongated body along the longitudinal axis, comprising: an opening comprising a tip outer diameter smaller than the elongated body outer diameter; a neck proximal of the opening comprising the tip outer diameter; and a shoulder disposed between the neck and the elongated body; a central lumen extending from the proximal end to the distal end along the longitudinal axis through the tip; and a bump extending longitudinally from the elongated body to proximate the opening and being partially circumferentially attached to at least a portion of the neck and the elongated body, forming an asymmetric cross section of the distal end.
Clause 2. The catheter of Clause 1, wherein the asymmetric cross section comprises an outer diameter greater than an average inner diameter of an ophthalmic artery to prevent the catheter from entering the ophthalmic artery.
Clause 3. The catheter of Clause 1 or 2, wherein the asymmetric cross section comprises an outer diameter smaller than an average inner diameter of a cerebral vessel to allow the catheter to go through the cerebral vessel.
Clause 4. The catheter of any of preceding Clauses, further comprising: a side lumen disposed within a sidewall of the catheter extending from the proximal end to the distal end along the longitudinal axis and exiting through the tip, configured to receive a guidewire.
Clause 5. The catheter of any of preceding Clauses, wherein the central lumen comprises a uniform inner diameter.
Clause 6. A catheter, comprising: an elongated body comprising a proximal end, a distal end, a longitudinal axis, and an elongated body outer diameter; a tip disposed at the distal end of the elongated body along the longitudinal axis, comprising: an opening comprising a tip outer diameter smaller than the elongated body outer diameter; a neck proximal of the opening comprising the tip outer diameter; and a shoulder disposed between the neck and the elongated body; a central lumen extending from the proximal end to the distal end along the longitudinal axis through the tip; and at least one steering lumen extending within a sidewall of the catheter from the proximal end to the distal end along the longitudinal axis through the tip, the at least one steering lumen being closed proximate the opening.
Clause 7. The catheter of Clause 6, wherein the at least one steering lumen comprises a steering lumen opening on a circumferential tip outer surface proximate the opening.
Clause 8. The catheter of Clause 7, wherein the steering lumen opening extends substantially radially through the sidewall.
Clause 9. The catheter of Clause 6, wherein the at least one steering lumen comprises a sealed distal end proximate the opening.
Clause 10. The catheter of Clause 9, wherein the at least one steering lumen comprises at least one branch extending substantially radially inwardly with an inflated internal volume when its inside injected liquid is pressurized.
Clause 11. The catheter of any of preceding Clauses 6-10, wherein the catheter comprises at least two steering lumens symmetrically disposed about a full circumference of the catheter.
Clause 12. The catheter of any of preceding Clauses 6-11, wherein the catheter comprises at least four steering lumens.
Clause 13. The catheter of any of preceding Clauses 6-12, further comprising: a side lumen disposed within a sidewall of the catheter extending from the proximal end to the distal end along the longitudinal axis and exiting through the tip, configured to receive a guidewire.
Clause 14. The catheter of Clause 13, further comprising a guidewire incorporated within the side lumen.
Clause 15. The catheter of any of preceding Clauses 6-12, further comprising: a side lumen disposed within a sidewall of the catheter extending from the proximal end to the distal end along the longitudinal axis and exiting through the shoulder, configured to receive a guidewire.
Clause 16. The catheter of any of preceding Clauses 6-14, further comprising: an inflatable annular balloon being full circumferentially attached to at least a portion of the elongated body, or a portion of the tip.
Clause 17. The catheter of Clauses 16, wherein the inflatable annular balloon when inflated comprises an outer diameter greater than an average inner diameter of an ophthalmic artery to prevent the catheter from entering the ophthalmic artery.
Clause 18. The catheter of Clauses 16 or 17, wherein the outer diameter of the inflatable annular balloon when inflated is smaller than an average inner diameter of a cerebral vessel, to allow the catheter to go through the cerebral vessel.
Clause 19. A method for steering a catheter, the catheter comprising an elongated body, a tip, a central lumen and at least one steering lumen, the elongated body comprising a proximal end, a distal end, a longitudinal axis, and an elongated body outer diameter; the tip disposed at the distal end of the elongated body along the longitudinal axis, comprising: an opening comprising a tip outer diameter smaller than the elongated body outer diameter; a neck proximal of the opening comprising the tip outer diameter; and a shoulder disposed between the neck and the elongated body; the central lumen extending from the proximal end to the distal end along the longitudinal axis through the tip; and the at least one steering lumen extending within a sidewall of the catheter from the proximal end to the distal end along the longitudinal axis through the tip, the at least one steering lumen being closed proximate the opening, the method comprising the steps of: selecting a steering lumen from the at least one steering lumen; injecting a liquid into the selected steering lumen; pressurizing the selected steering lumen; and steering the tip of the catheter.
Clause 20. The method for steering a catheter of Clause 19, further comprising the step of: injecting saline into the selected steering lumen.

The descriptions contained herein are examples of embodiments of the invention and are not intended in any way to limit the scope of the invention. As described herein, the invention contemplates many variations and modifications of a catheter, including attaching a bump and/or attaching an inflatable annular balloon to a catheter, configuring at least one steering lumen inside the catheter to enhance its navigability to a certain extent. Also, an additional side lumen may be configurated allowing the catheter to have a built in or incorporated guidewire beforehand. Various combinations of those exemplary implementations may be conceived and appropriately designed. Modifications and variations apparent to those having skilled in the pertinent art according to the teachings of this disclosure are intended to be within the scope of the claims which follow.

What is claimed is:

1. A catheter comprising:
an elongated body comprising a proximal end, a distal end, a longitudinal axis, and an elongated body outer diameter;
a tip disposed at the distal end of the elongated body along the longitudinal axis, comprising:
an opening comprising a tip outer diameter smaller than the elongated body outer diameter;
a neck proximal of the opening comprising the tip outer diameter; and
a shoulder disposed between the neck and the elongated body;
a central lumen extending from the proximal end to the distal end along the longitudinal axis through the tip; and
a bump extending longitudinally from the elongated body to proximate the opening and being partially circumferentially attached to at least a portion of the neck and the elongated body, forming an asymmetric cross section of the distal end.

2. The catheter of claim 1, wherein the asymmetric cross section comprises an outer diameter greater than an average inner diameter of an ophthalmic artery to prevent the catheter from entering the ophthalmic artery.

3. The catheter of claim 2, wherein the outer diameter of the asymmetric cross section is smaller than an average inner diameter of a cerebral vessel to allow the catheter to go through the cerebral vessel.

4. The catheter of claim 1, further comprising:
a side lumen disposed within a sidewall of the catheter extending from the proximal end to the distal end along the longitudinal axis and exiting through the tip, configured to receive a guidewire.

5. The catheter of claim 1, wherein the central lumen comprises a uniform inner diameter.

6. A catheter, comprising:
an elongated body comprising a proximal end, a distal end, a longitudinal axis, and an elongated body outer diameter;
a tip disposed at the distal end of the elongated body along the longitudinal axis, comprising:
an opening comprising a tip outer diameter smaller than the elongated body outer diameter;
a neck proximal of the opening comprising the tip outer diameter; and
a shoulder disposed between the neck and the elongated body;
a central lumen extending from the proximal end to the distal end along the longitudinal axis through the tip; and
at least one steering lumen extending within a sidewall of the catheter from the proximal end to the distal end along the longitudinal axis through the tip, the at least one steering lumen being closed proximate the opening.

7. The catheter of claim 6, wherein the at least one steering lumen comprises a steering lumen opening on a circumferential tip outer surface proximate the opening.

8. The catheter of claim 7, wherein the steering lumen opening extends substantially radially through the sidewall.

9. The catheter of claim 6, wherein the at least one steering lumen comprises a sealed distal end proximate the opening.

10. The catheter of claim 9, wherein the at least one steering lumen comprises at least one branch extending substantially radially inwardly with an inflated internal volume when its inside injected liquid is pressurized.

11. The catheter of claim 6, wherein the catheter comprises at least two steering lumens symmetrically disposed about a full circumference of the catheter.

12. The catheter of claim 11, wherein the catheter comprises at least four steering lumens.

13. The catheter of claim 6, further comprising:
a side lumen disposed within a sidewall of the catheter extending from the proximal end to the distal end along the longitudinal axis and exiting through the tip, configured to receive a guidewire.

14. The catheter of claim 13, further comprising a guidewire incorporated within the side lumen.

15. The catheter of claim 6, further comprising:
a side lumen disposed within a sidewall of the catheter extending from the proximal end to the distal end along the longitudinal axis and exiting through the shoulder, configured to receive a guidewire.

16. The catheter of claim 6, further comprising:
an inflatable annular balloon being full circumferentially attached to at least a portion of the elongated body, or a portion of the tip.

17. The catheter of claim 16, wherein the inflatable annular balloon when inflated comprises an outer diameter greater than an average inner diameter of an ophthalmic artery to prevent the catheter from entering the ophthalmic artery.

18. The catheter of claim 17, wherein the outer diameter of the inflatable annular balloon when inflated is smaller than an average inner diameter of a cerebral vessel, to allow the catheter to go through the cerebral vessel.

19. A method for steering a catheter, the catheter comprising an elongated body, a tip, a central lumen and at least one steering lumen, the elongated body comprising a proximal end, a distal end, a longitudinal axis, and an elongated body outer diameter; the tip disposed at the distal end of the elongated body along the longitudinal axis, comprising: an opening comprising a tip outer diameter smaller than the elongated body outer diameter; a neck proximal of the opening comprising the tip outer diameter; and a shoulder disposed between the neck and the elongated body; the central lumen extending from the proximal end to the distal end along the longitudinal axis through the tip; and the at least one steering lumen extending within a sidewall of the catheter from the proximal end to the distal end along the longitudinal axis through the tip, the at least one steering lumen being closed proximate the opening, the method comprising the steps of:
selecting a steering lumen from the at least one steering lumen;
injecting a liquid into the selected steering lumen;
pressurizing the selected steering lumen; and
steering the tip of the catheter.

20. The method for steering a catheter of claim 19, further comprising the step of:
injecting saline into the selected steering lumen.
